(19) United States
(12) Patent Application Publication
(10) Pub. No.: US 2015/0305927 A1
(43) Pub. Date: Oct. 29, 2015

(54) SURGICAL TOOLS AND SYSTEMS FOR CORNEAL TATTOOING AND RELATED METHODS

(71) Applicant: Wake Forest University Health Sciences, Winston-Salem, NC (US)

(72) Inventor: Keith Andrew Walter, Lewisville, NC (US)

(21) Appl. No.: 14/694,552

(22) Filed: Apr. 23, 2015

Related U.S. Application Data
(60) Provisional application No. 61/984,321, filed on Apr. 25, 2014.
(60) Provisional application No. 62/015,064, filed on Jun. 20, 2014.

(51) Int. Cl. A61F 9/00; A61M 37/00; A61F 9/008

(52) U.S. Cl. CPC A61F 9/0008; A61F 9/00836; A61M 37/0084; A61M 37/0076; A61F 9/00838

(57) ABSTRACT

Devices, systems, surgical kits and methods for corneal surgery, preferably using femto-laser assisted, corneal tattooing. The corneal tattoos are formed with one or more viscous opaque substances such as pasty substances of different colors to match eye colors of different patients.

### RELATED APPLICATIONS

This application claims the benefit of and priority to U.S. Provisional Application Ser. No. 61/984,321, filed Apr. 25, 2014 and U.S. Provisional Application Ser. No. 62/015,064, filed Jun. 20, 2014, the contents of which are hereby incorporated by reference as if recited in full herein.

### FIELD OF THE INVENTION

This invention relates to tools for use in ophthalmic surgeries such as, for example, surgeries for patients with aniridia, iris trauma and other disorders or injury and potentially cosmetic change of iris or eye color.

### BACKGROUND OF THE INVENTION

Conventional corneal tattooing for medical purposes involves the use of a 30-gauge needle to apply titanium or iron dioxide pigment to the cornea using small punctures to the eye. It typically takes 20 to 30 minutes to make between 200 to 1,000 small needle punctures to place the dye. The procedure is frequently incomplete, with a high re-treatment rate, and it can be painful. The dye coverage applied by the direct injections may also be incomplete.

### SUMMARY OF EMBODIMENTS OF THE INVENTION

Embodiments of the invention provide methods and related surgical tools suitable for ophthalmic corneal tattoo surgery.

Embodiments of the invention are directed to methods of performing corneal tattoo surgery. The methods can include: selecting an incision location, size and depth for a corneal flap, channel or pocket; then transmitting laser pulses to a lens of an eye of a patient to generate the corneal flap, channel or pocket; and applying a corneal tattoo substance to an internal corneal tissue and/or layer.

The corneal tattoo can be applied to the corneal layer to be in-line with a region of an iris over a pupil.

The applying can include manually pushing the corneal tattoo substance downward onto the cornea layer using a surgical tool to form a continuous layer or layers over a target area to form the corneal tattoo.

The selecting can be carried out by a user selecting a ring inner and outer diameter for a channel via a user interface in communication with a Femtosecond laser optical system. The transmitting laser pulses can be carried out by the Femtosecond laser optical system.

The applying the corneal tattoo substance is carried out using at least one opaque, viscous dye having a clinically effective life of at least five years.

The method can also include applying a striation pattern using a secondary dye onto the applied corneal tattoo to thereby generate a more natural looking iris for the corneal tattoo.

The method can include, before the applying step, selecting a color corresponding to a primary color of the iris of the patient.

The corneal tattoo can be applied at a corneal depth of between 150 μm to 300 μm.

The selecting and transmitting steps can be carried out to generate an annular corneal channel. The annular corneal channel can have an inner diameter of between 4-5 mm and an outer diameter that is between 1 mm to 3 mm larger than the inner diameter.

The patient can be treated for aniridia or iris trauma.

Other embodiments are directed to surgical tools for corneal tattooing. The tools can include a handle with a shaft that is attached to a corneal applicator, the corneal applicator sized and configured to push against an internal (e.g., dissected) corneal layer of a cornea to distribute a viscous, opaque corneal tattoo substance.

The surgical tool can be in combination with at least one container of an opaque, viscous medical grade corneal tattoo substance.

The corneal applicator can include at least one depression or well for holding a quantity of the corneal tattoo substance.

The corneal applicator can have a thickness between 1 micron and 5 mm and extends off only a single side of the shaft.

The surgical tool can include a plurality of vertically extending through holes in a depression or well.

The corneal applicator can have a radially extending length and a circumferentially extending width associated with an outer perimeter edge. The outer perimeter edge can extend away from the shaft at an angle between 10-180 degrees.

The corneal applicator can be arcuate with opposing circumferentially spaced apart first and second ends. The first end can be attached to the shaft and the second end can be a free end. The free end can reside circumferentially spaced apart from the shaft at between 90-180 degrees.

Yet other embodiments are directed to surgical kits or sets of components for treating an eye. The sets/kits can include: a surgical tool with a corneal applicator; and at least one container of a medical grade corneal tattoo substance. The corneal tattoo substance can be formulated to be opaque and viscous.

The kit or set can include a striation tool configured to allow a user to apply a striation pattern over an opaque primary corneal tattoo that covers a continuous surface area over a target treatment site of a corneal layer to thereby form a more natural iris pattern for a corneal tattoo.

The kit and/or set of tools can include a plurality of containers of the medical grade corneal tattoo substance, each of a different color.

The corneal applicator can either (a) have a radially extending length and a circumferentially extending width associated with an outer perimeter edge, wherein the outer perimeter edge extends away from the shaft at an angle between 10-180 degrees or (b) be arcuate with opposing circumferentially spaced apart first and second ends. The first end can be attached to the shaft and the second end can be a free end that resides circumferentially spaced apart from the shaft at between 90-180 degrees.

Still other embodiments are directed to a femtosecond laser surgical system for eye treatments. The system includes: a femtosecond laser; a controller in communication with the laser, the controller comprising or in communication with a corneal tattoo module, wherein the corneal tattoo module allows a user to select laser inputs including a corneal channel depth between 100 and 300 μm; and a display having a user interface in communication with the controller. The user interface can allow a user to overlay a virtual ring over an image of an eye of a patient to identify a desired a ring size that will allow access to an internal corneal layer (which may be in-line with a region of an iris over a pupil).

The system can include an imaging system in communication with the controller. The system can include a color match module for evaluating an eye color of a patient based on an image taken using the imaging system and providing color information to a user for selecting an appropriate product or color for a corneal tattoo substance.

Some embodiments of the invention provide methods, laser systems and tools that are configured to create a corneal tattoo to form a pinhole effect in the central cornea by creating a very small aperture area surrounded by an opaque area of tattoo substance (pigment/dye) with an outer diameter of 1-4 mm.

The foregoing and other objects and aspects of the present invention are explained in detail in the specification set forth below.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic illustration of an eye.

FIG. 2A is a greatly enlarged partial view of an eye illustrating a channel access to a target corneal surface according to embodiments of the present invention.

FIG. 2B is a greatly enlarged partial view of an eye illustrating a flap access to a target corneal surface according to embodiments of the present invention.

FIGS. 3A-3D are digital photographs of eyes having injuries for treatment according to embodiments of the invention.

FIGS. 3E-3H are digital photographs of the eyes shown in FIGS. 3A-3D with applied corneal tattoos according to embodiments of the present invention.

FIGS. 3I and 3J illustrate that the corneal tattoo can be used to create a pinhole effect in a cornea according to embodiments of the present invention.

FIGS. 4A and 4B are partial views of a display window with a user interface (UI) for allowing a user to select treatment parameters for a Femtosecond surgical/optical laser system according to embodiments of the present invention.

FIG. 5 is a schematic illustration of an optical therapy system with a laser and corneal tattoo module according to embodiments of the present invention.

FIG. 6A is a greatly enlarged top perspective view of an exemplary surgical corneal tattoo dye applicator tool according to embodiments of the present invention.

FIG. 6B is a side perspective view of the surgical tool shown in FIG. 6A.

FIG. 6C is an end perspective view of the surgical tool shown in FIG. 6A.

FIG. 7A is a schematic illustration of a surgical tool set and/or kit according to embodiments of the present invention.

FIG. 7B is a schematic illustration of another surgical tool set and/or kit (or subset of such) according to embodiments of the present invention

FIG. 8 is a top perspective view of a sterile mixing board for customizing dye colors applied to an eye of a patient according to embodiments of the present invention.

FIG. 9 is a schematic illustration of an optical treatment system according to embodiments of the present invention.

FIG. 10 is a schematic illustration of surgical tools that can be used to form a corneal tattoo according to embodiments of the present invention.

FIG. 11 is a greatly enlarged top perspective view of another exemplary corneal tattoo dye applicator according to embodiments of the present invention.

FIG. 12 is a flow chart of operations that can be carried out to apply a corneal tattoo according to embodiments of the present invention.

### DESCRIPTION OF EMBODIMENTS OF THE INVENTION

The present invention will now be described more fully hereinafter with reference to the accompanying figures, in which embodiments of the invention are shown. This invention may, however, be embodied in many different forms and should not be construed as limited to the embodiments set forth herein. Like numbers refer to like elements throughout. The term “Fig.” (whether in all capital letters or not) is used interchangeably with the word “Figure” as an abbreviation thereof in the specification and drawings. In the figures, certain layers, components or features may be exaggerated for clarity, and broken lines illustrate optional features or operations unless specified otherwise. In addition, the sequence of operations (or steps) is not limited to the order presented in the figures and/or claims unless specifically indicated otherwise. In the drawings, the thickness of lines, layers, features, components and/or regions may be exaggerated for clarity and broken lines illustrate optional features or operations, unless specified otherwise.

The terminology used herein is for the purpose of describing particular embodiments only and is not intended to be limiting of the invention. As used herein, the singular forms, “a”, “an” and “the” are intended to include the plural forms as well, unless the context clearly indicates otherwise. It will be further understood that the terms “comprises,” “comprising,” “includes,” and/or “including” when used in this specification, specify the presence of stated features, regions, steps, operations, elements, and/or components, but do not preclude the presence or addition of one or more other features, regions, steps, operations, elements, components, and/or groups thereof.

It will be understood that when a feature, such as a layer, region or substrate, is referred to as being “on” another feature or element, it can be directly on the other feature or element or intervening features and/or elements may also be present. In contrast, when an element is referred to as being “directly on” another feature or element, there are no intervening elements present. It will also be understood that, when a feature or element is referred to as being “connected”, “attached” or “coupled” to another feature or element, it can be directly connected, attached or coupled to the other element or intervening elements may be present. In contrast, when a feature or element is referred to as being “directly connected”, “directly attached” or “directly coupled” to another element, there are no intervening elements present. Although described or shown with respect to one embodiment, the features so described or shown can apply to other embodiments.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

The word “about” means that the size or amount referred to can vary from the particular amount, typically by +/−10%.

The term “sterile” means that the device meets medical cleanliness standards for surgical use.

As used herein, the term “corneal tattoo” refers to a coating of an opaque or substantially opaque substance (e.g., a pigment) applied to the cornea at a sub-exterior surface depth at a location that is typically in line with a portion of a properly functioning or formed iris above the pupil to resolve, repair or improve vision problems and/or glare. The term “substantially” with respect to “opaque” means that light transmission associated with normal ambient room lighting or natural light is reduced by at least 90% relative to a surface without such a substance.

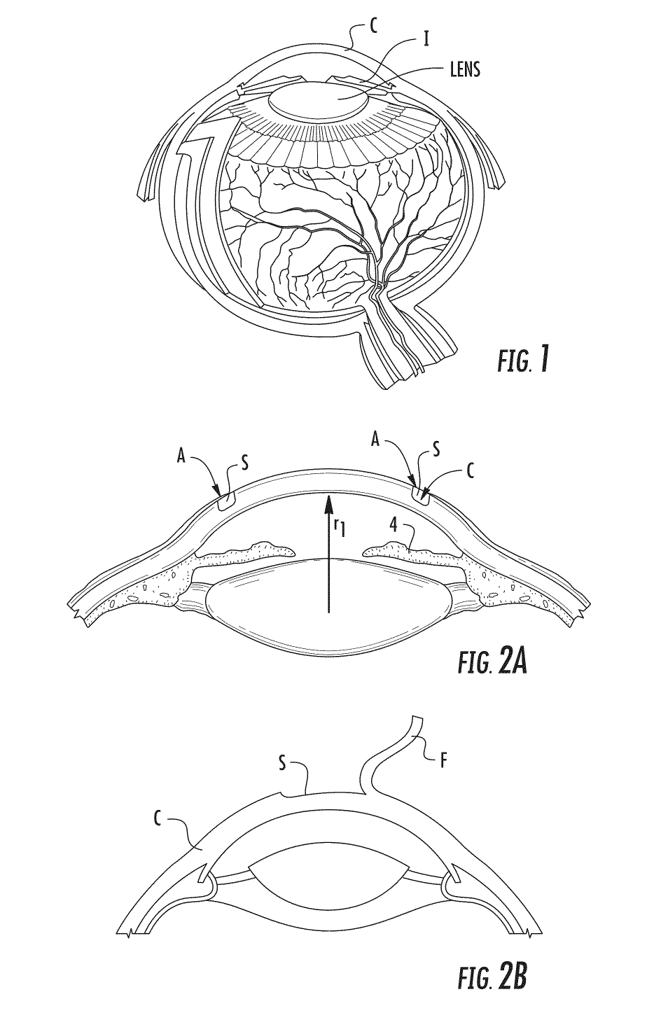

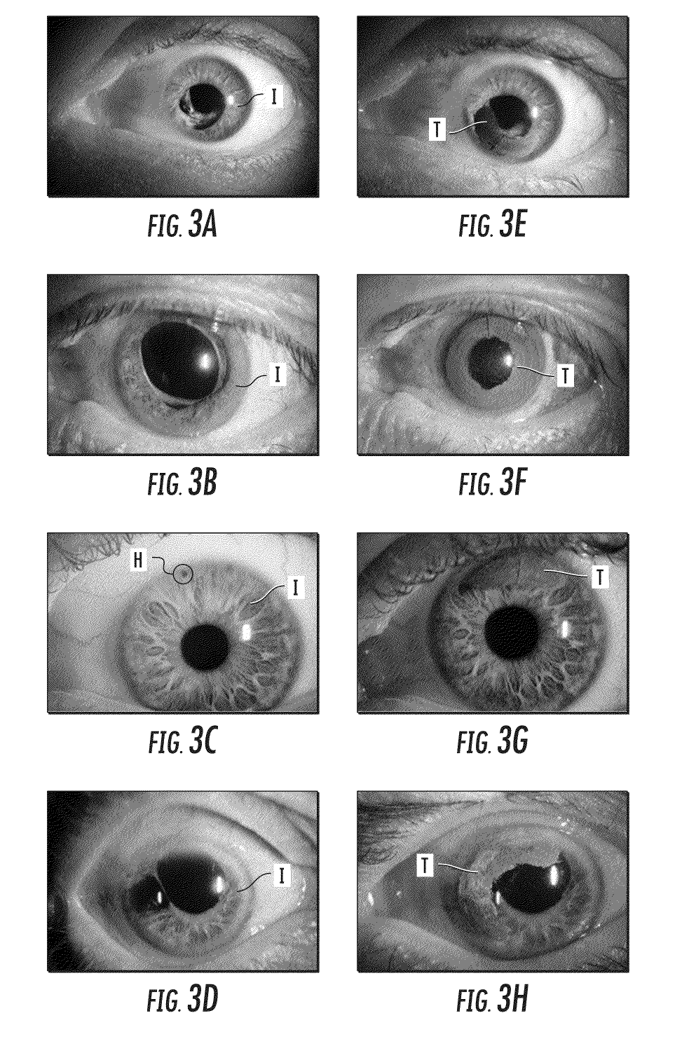

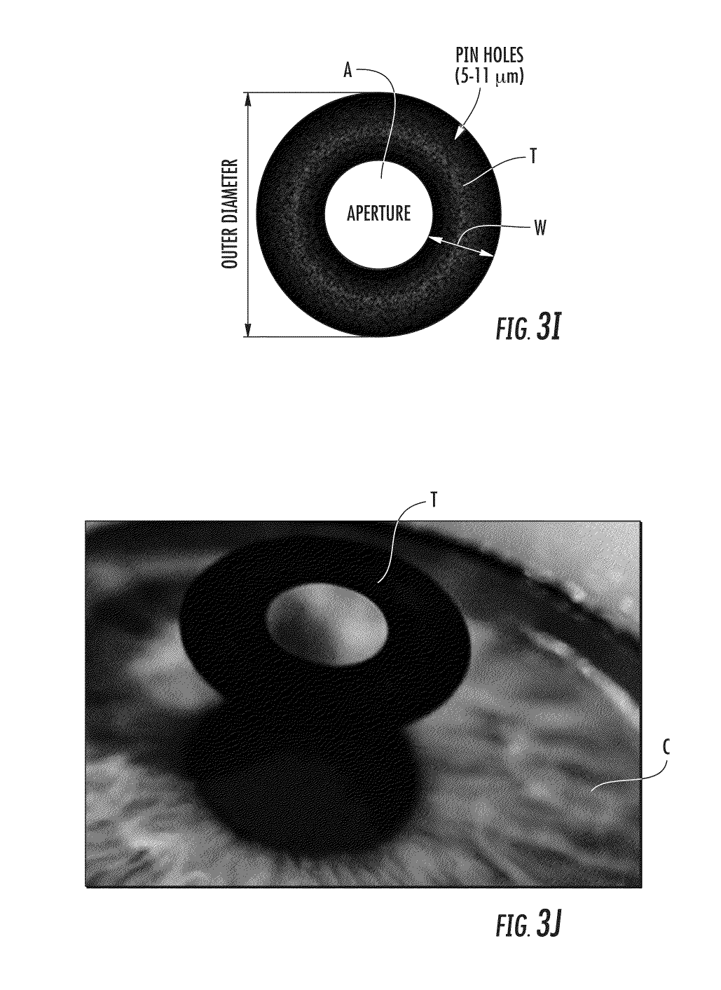

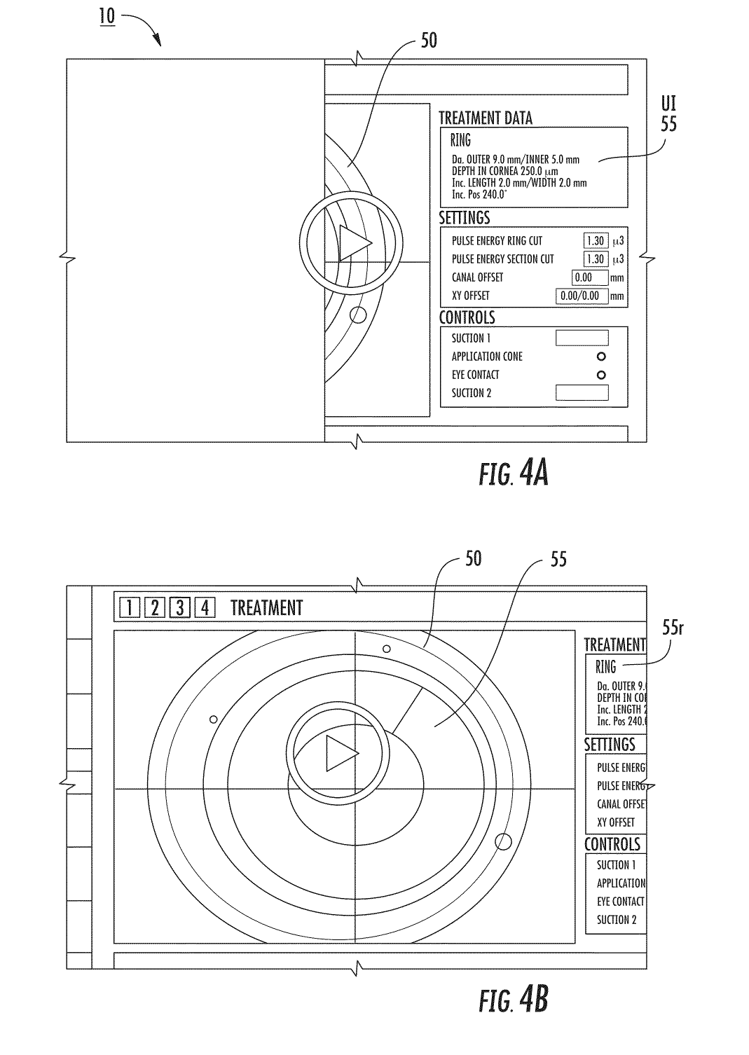

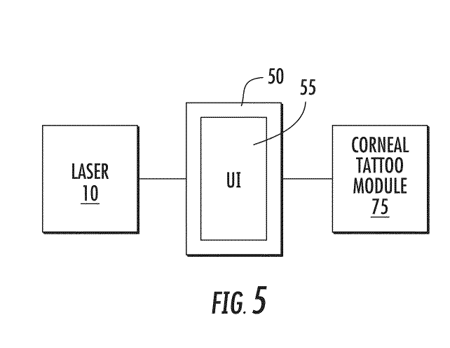

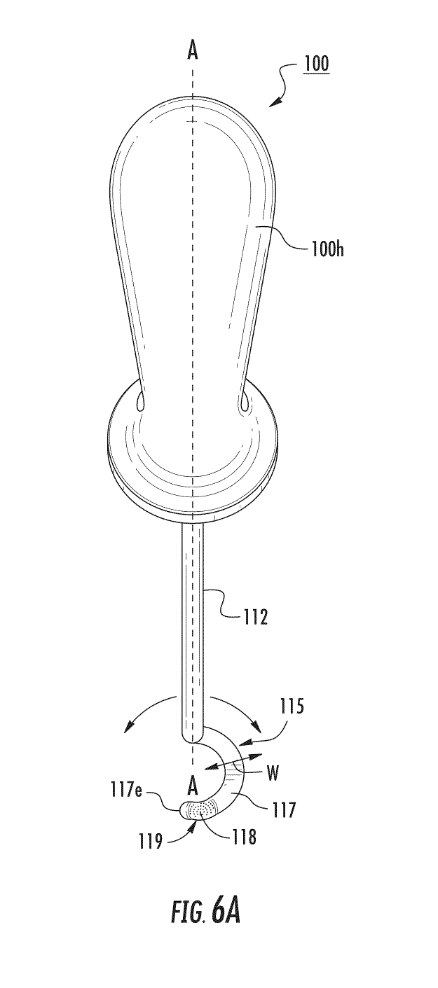

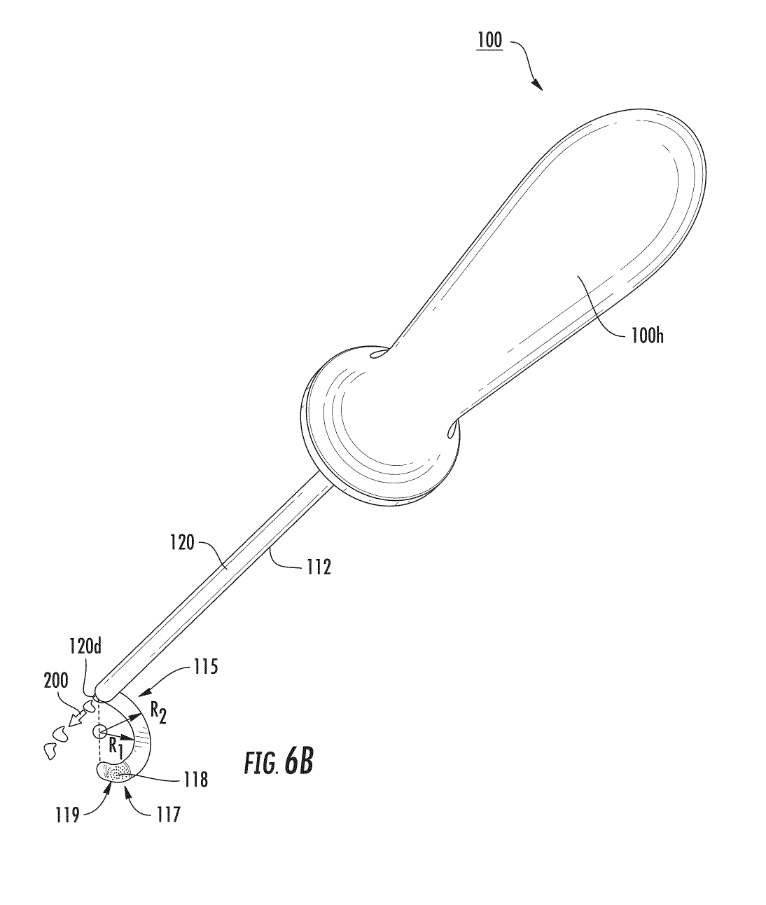

FIG. 1 illustrates components of the eye. A corneal access channel A (FIG. 2A) or flap F (FIG. 2B) can be made in a cornea C to provide access to a target corneal surface S. Laser created access channels A to create pockets are well known. See, e.g., U.S. Patent Application Publication 2012/0116505 and U.S. Pat. No. 8,123,803, the contents of which are hereby incorporated by reference as if recited in full herein. FIG. 2A illustrates that a corneal access channel A can be created in a cornea C to allow access to a corneal surface S for applying a corneal tattoo T (FIGS. 3E, 3F, 3G and 3H) to a desired portion of the corneal surface. The flap F can be generated using a hand-held device known as a microkeratome or blade. However, it is preferred that the access channel A or flap F (when used) be generated bladelessly, e.g., directly using a suitable laser such as a high energy laser (femtosecond laser) instead of a blade.

The substance used to form the corneal tattoo T can comprise a medical grade substance, preferably one that has a long life so as to avoid undue numbers of repeat procedures. The substance can have a clinically effective life, once applied, that is at least 5 years, typically between about 5 years to about 100 years or more. The substance can comprise an ink and/or insoluble pigment such as one or more of an Indian ink, iron oxide and/or titanium dioxide. The substance may alternatively or additionally comprise a chemical dye that may comprise metal salts such as gold chloride, silver nitrate, platinum chloride or the like. The substance can be viscous and/or paste-like so as to adhere or remain in a desired spot or treatment area on the cornea surface S. The term “viscous” when referring to the corneal tattoo substance means that the substance has a sufficiently thick consistency so that it does not flow at body temperature but can be spread over and/or within corneal tissue to cover a target treatment site. The substance can be gelatinous or pasty, for example.

In preferred embodiments, a titanium dioxide pigment of a desired color (or black or white) or a titanium dioxide pigment mixture of a plurality of colors can be applied to correspond with a color associated with a respective patient's iris. The substance can be applied in one or more layers. Where different layers are used, different layers may comprise different colors of the same or different substances.

FIGS. 3A-3H show examples of “before” (left column) and “after”(right column) images with the after illustrating the corneal tattoo T that has been applied according to embodiments of the present invention. FIGS. 3A and 3D are examples of traumatic injuries. FIG. 3B is an example of floppy iris syndrome. FIG. 3C illustrates a pin hole perforation H from iridotomoy holes made by a YAG laser prior to implantable contact lenses (small black dot at about 11:30 on the iris).

FIGS. 3I and 3J illustrate that the corneal tattoo T can be used to form a pinhole effect in the cornea similar to the Kamra™ inlay. The tattoo T can surround a small center open aperture A of between about 0.5 mm to about 2 mm, typically. The corneal tattoo T can have an outer diameter that is between 1-4 mm, such as about 1 mm, about 1.5 mm, about 2 mm, about 2.5 mm, about 3 mm, about 3.5 mm and about 4 mm. In some embodiments, the corneal tattoo T can have an annular shape with a radial width of between 1-2 mm. In particular embodiments, the tattoo T can be formed to have a 3.8-mm outer diameter about a 1.6-mm central aperture A. The annular shaped corneal tattoo T can be used with thousands of microperforations (e.g., 8,400 micro-perforations) that can be the same size or vary in size from 5 to 11 μm. The principle of the corneal tattoo's T function is similar to that of the small-aperture effect in an f-stop camera; it has minimal effect on distance image quality but improves intermediate and near image quality.

The corneal tattoo T can be formed in situ directly on the cornea using an appropriate surgical tool and corneal tattoo substance, e.g., pasty pigment. The surgical laser systems may be configured to project a shaped light guide that form a virtual annular template at a desired corneal tattoo site. It is alternatively contemplated that the corneal T can be pre-formed as a semi-solid substance with a binder, substrate, film, or other carrier that can remain, dissolve or be removed after or during the implantation, so that the annular tattoo retains or takes the annular shape while it is implanted.

It is contemplated that a corneal tattoo T may be an alternative to the corneal inlay, intended to improve near vision without compromising distance vision in emmetropic presbyopes and possibly non-emmetropes as well such as the Kamra™ inlay from AcuFocus in Irvine, Calif., which uses the pinhole principle to increase depth of field. See, Waring GO 4th. Correction of presbyopia with a small aperture corneal inlay. J Refract Surg 2011; 27:842-5; Seyeddain O, Hohensinn M, et al. Small-aperture corneal inlay for the correction of presbyopia: 3-year follow-up. J Cataract Refract Surg 2012; 38:35-45; Chayet A, Garza E B. Combined hydrogel inlay and laser in situ keratomileusis to compensate for presbyopia in hyperopic patients: One-year safety and efficacy. J Cataract Refract Surg 2013; 39:1713-21; Garza E B, Gomez S, Chayet A, Dishier J. One-year safety and efficacy results of a hydrogel inlay to improve near vision in patients with emmetropic presbyopia. J Refract Surg 2013; 29:166-72; and Limnopoulou A N, Bouzoukis D I, et al. Visual outcomes and safety of a refractive corneal inlay for presbyopia using femtosecond laser. J Refract Surg 2013; 29:12-8. The contents of these documents are hereby incorporated by reference as if recited in full herein.

In preferred embodiments, access to the target corneal surface S is created using a Femtosecond laser, e.g., “Femto-Assisted Corneal Tattooing.” As is well known to those of skill in the art, Femtosecond lasers have been proposed for use in cataract surgeries. See, e.g., Nagy et al, Initial clinical evaluation of an intraocular femtosecond laser in cataract surgery. J Refract Surg 2009; 25:1053-60. See also, Ella Faktorovich, Femtodynamics: A Guide to Laser Settings and Procedure Techniques to Optimize Outcomes with Femtosecond Lasers, (Slack Inco., 1^{st} edition, 2009). The contents of these documents are hereby incorporated by reference as if recited in full herein.

The inventor has found that Femtosecond laser-assisted corneal tattooing has shown good early results for patients with aniridia, iris trauma and other disorders.

The Femtosecond laser allows the surgeon to make a custom, laser generated corneal pocket or channel (or flap) at any location.

In some embodiments, corneal channels having a depth of about 150 μm to about 300 μm can be generated and the corneal tattoo T can be applied at this depth. FIGS. 3E-3H illustrate corneal tattooing T with relatively good cosmetic results and rapid recovery. The corneal tattoos can treat patients with traumatic injury and visual side effects from ICL (STAAR Surgical) implantation, and other problems.

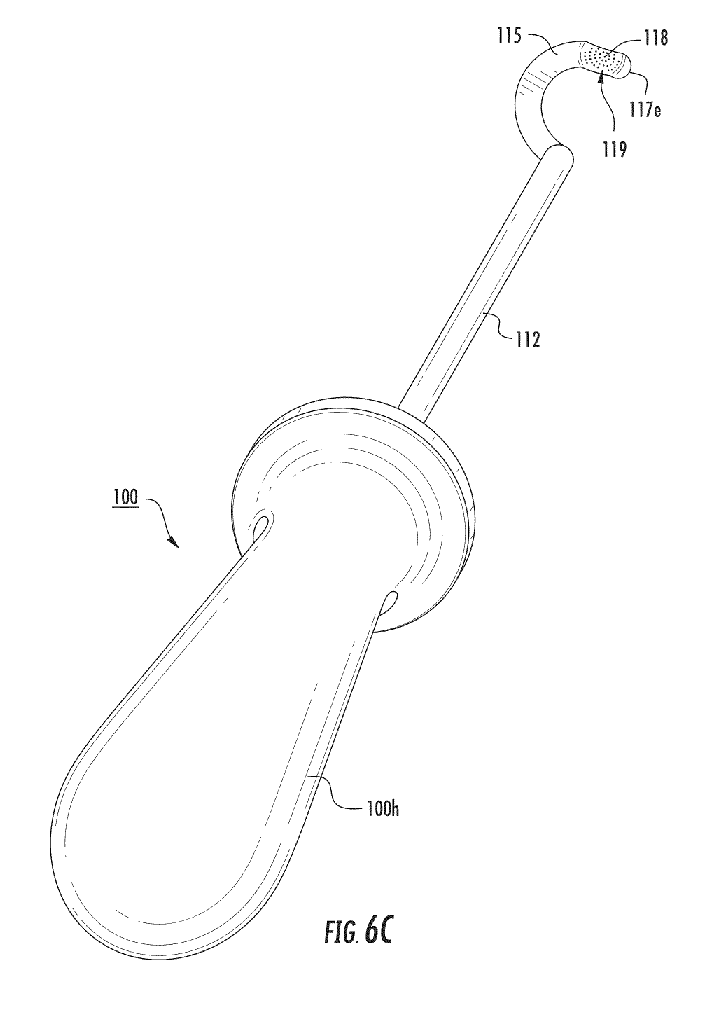

As shown in FIGS. 4A and 4B, the surgeon can use a Femtosecond surgical system 10 with a user interface (UI) 55 on a display 50 that provides operational input selections for creating the corneal channel A. The operational input selection can include a GUI screen that provides a ring segment input 55r to programmatically provide a selection of channels of suitable depths for corneal tattoos and/or to create a partial or complete Intacs channel A at a patient-specific location appropriate for treatment using a corneal tattoo T.

The Femtosecond laser system 10 can include a corneal tattoo module 75 that can be configured to provide a selection of shapes and sizes that are optimized for use to create appropriate corneal tattoos at appropriate controlled depths, typically limited to between about 100 μm to about 300 μm, more typically between about 150 μm to about 250 μm, Examples of currently available femtosecond laser optical systems are believed to include Alcon LenSx or Alcon FS200 (Alcon Laboratories, Ft Worth, Tex., USA), OptiMedica Catalys (Optimedica Corp, California, USA), LensAR (LensAR Inc, Florida, USA), the FS IntraLase™ FS Laser system from Abbott Medical Optics, Technolas (Technolas Perfect Vision GmbH, Germany) as well as femtosecond laser systems from Ziemer or Bausch and Lomb. The laser systems 10 typically include an anterior segment imaging system, patient interface and Femtosecond laser to image, calculate and deliver the laser pulses.

Traditional LASIK flaps were generally ranged in thickness from 140 to 180 microns. This is about one third of the total corneal thickness, given that the average normal cornea is about 540 microns in thickness. Newer types of microkeratomes and IntraLase™ lasers can create thinner flaps and channels. Many LASIK surgeons prefer a flap thickness between 100 and 130 microns. The flap F for corneal tattooing may be made at a greater depth than for cataract surgeries as noted by the examples of depths described above. The annular corneal channels A can have an inner diameter between about 4-5 mm and the outer diameter can be 1 mm to about 4 mm larger. The outer diameter is typically between about 6 to 10 mm, such as 6 mm, 6.5 mm, 7 mm, 7.5 mm, 8 mm, 8.5 mm, 9 mm, 9.5 min or 10 mm.

Once the corneal pocket/channel A or flap F is created at a suitable location for a corneal tattoo T, which can be patient specific as to size, shape and depth, the tattoo substance can be applied. Unlike conventional multiple injection needle application, a wider area can be directly covered using one or more applicators for a coating that has a continuous surface area.

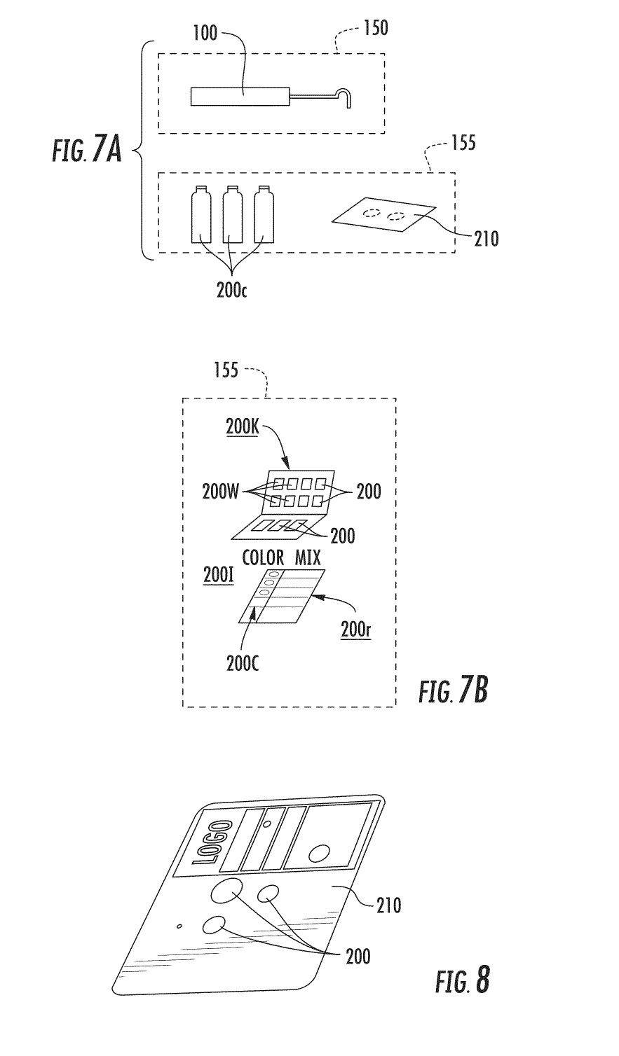

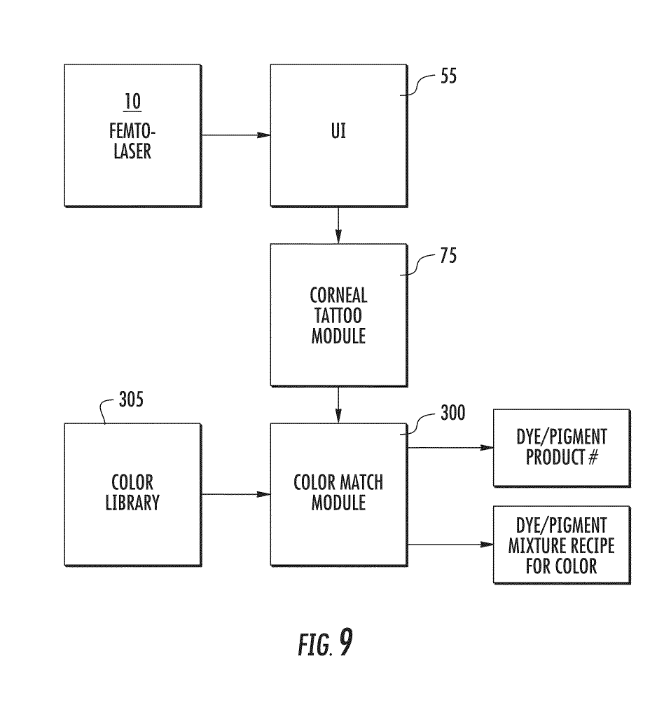

FIGS. 6A-6C illustrate an example of a surgical tool 100 that can be used to place, spread and/or push the tattoo substance onto the target corneal surface S. The tool 100 can be a manual tool with a handle 100h and a distal end 115. The distal end 115 can be configured to lightly push downward into the cornea with the tattoo substance to spread, distribute or otherwise substantially evenly apply the tattoo substance to the target treatment area. As shown, the distal end 115 of the tool 100 can have a planar bottom 117 that contacts the tattoo substance. The bottom 117 can be oriented to be orthogonal to a plane extending along the axis A-A of the shaft 112 connecting the distal end 115 to the handle 100h. The

The bottom 117 can comprise one or more apertures 118, typically a plurality of small apertures on a leading free end thereof, to allow fluid to travel therethrough. The apertures 118 can be held in at least one well 119 (e.g., depression or recess in a substrate or other material on or in the blade or distal end 115). The small apertures can be in the sub-millimeter, e.g., micron and/or nanometer size range.

The bottom 117 can have a thin thickness that can be in the nanometer, micron or millimeter range, typically between about 1 micron to about 10 mm, more typically between about 1 micron and 5 mm, such as about 10 microns, about 20 microns, about 30 microns, about 40 microns, about 50 microns, about 60 microns, about 70 microns, about 80 microns, about 90 microns, about 0.1 mm, about 0.5 mm, about 1 mm, about 2 mm, about 3 mm, about 4 mm, about 5 mm or any number between the noted range. Other suitable thicknesses may be used.

The distal end 115 can have an arcuate or semi-circular configuration as shown and can extend from about 30 to about 90 degrees, shown as about 90 degrees. The outer (free) end of the bottom 117e can be curved and/or tapered. However, the distal end 115 and/or bottom surface 117 can have other configurations.

The width of the bottom 117 can be between about 0.5 mm and about 15 mm, typically between about 0.5 mm and 5 mm, such as about 0.6 mm, about 0.7 mm, about 0.8 mm, about 0.9 mm, about 1 mm, about 1.5 mm, about 2 mm, about 2.5 mm, about 3 mm, about 3.5 mm, about 4 mm, about 4.5 mm, and about 5 mm.

FIG. 6B shows that the bottom 117 can be arcuate as discussed and may have an inner radius of curvature R1 and an outer radius of curvature R2, where R2>R1 and the difference R2-R1 defines the width of the tool thereat. The radius of curvature can be measured from a virtual centerline of an open space that is configured to be centered over a pupil when used to apply the tattoo substance. The inner radius of curvature R1 can be between 2 mm to about 4 mm (e.g., allowing an open diameter of at least 4 mm) so that there is a free open center that extends outside the center of the cornea when the tool 100 is rotated while held centered over the pupil. The arcuate end can extend in either a clockwise or a counterclockwise direction.

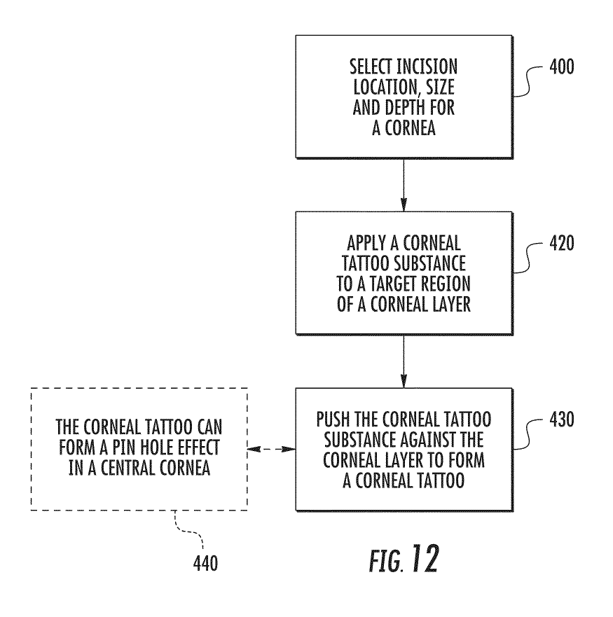

FIG. 11 illustrates another embodiment of the tool 100. In this embodiment, the distal end 115′ has a bottom 117 with at least one well 119 that can have a solid tattoo substance containment surface (as shown) or may optionally have one or more apertures. The at least one well 119 can hold the substance 200. The distal end 115 can be configured to have an arcuate body 115b with a width, typically a radius R2, that extends outwardly from one side of the shaft 112. The radius R2 can be between about 0.5 mm and 8 mm, typically between about 2 mm and 4 mm.

The bottom 117 can have a neck portion 117n that can have straight parallel sides. The neck portion 117n can be attached to the shaft 112. The neck portion 117n can extend a distance D₁ before merging into radially extending sides of the arcuate body segment 115b. In some embodiments, the distance D₁ can be about 2 mm or less, such as between about 10 microns and about 2 mm, typically between about 100 microns and 1 mm.

The arcuate body 115b can have an angular extension α of between about 10-180 degrees, typically between about 10-90 degrees, such as about 10 degrees, about 15 degrees, about 20 degrees, about 25 degrees, about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees and about 90 degrees.

The at least one well 119 can have a distal end portion that is wider than a proximal end portion (the proximal end portion residing closer to the shaft 112). The at least one well 119 can be etched, machined, stamped, molded or otherwise formed in the distal end 115 of the tool 100. The at least one well 119 may also be formed using a cooperating material such as a wax on the upper surface of the distal end/bottom 117 (not shown).

In some embodiments, the distal end 115 can have a plurality of adjacent, radially extending wells 119 as shown. The wells 119 can have the same depth or different depths. The wells 119 can have different sizes and/or shapes or all can have the same shape and size.

The at least one well 119 can have a shallow depth that is typically between 1 nanometer and about 10 millimeters (mm). The well depth can be under 1 mm. The depth can be in the micron and/or nanometer size range, such as between 1 nanometer and 100 microns, or between 10 nanometers and 10 microns, for example.

The tool 100 can be used to directly apply the tattoo substance 200, e.g., by dipping, spraying, pouring, injecting and/or coating a top and/or bottom surface 117 of the tool 100 and/or holding the tattoo substance 200 in a well 119 on the distal end portion 115.

The distal end 115 of the tool 100 can be placed on the target corneal tissue and the handle 100h and/or shaft 112 can be rotated to rotate the distal end of the tool 115 about the pupil.

In some embodiments the tool 100 can be configured with at least one well (e.g., a depression or recess in a substrate or other material in or on the blade or bottom 117) 119 that can hold the tattoo substance (e.g., dye or pigment). The at least one well 119 may optionally include at least one small hole in the bottom or floor of the well to aid in the distribution of the tattoo substance but the well may dispense the substance without requiring such a through aperture or hole.

In other embodiments, the tattoo substance can be injected with a separate tool to overlie a target portion of the surface area of the corneal tissue S, then the tool 100 can be used to spread the substance to provide a continuous coating or coverage. The initial applicator (e.g., injector) can comprise a device with a nozzle such as a syringe or needle without requiring direct contact (e.g., no piercing or puncturing).

As shown schematically in FIG. 6B, the tool 100 can be configured to also hold and discharge a tattoo substance 200 through a flow path 120 extending therein. The tattoo substance 200 can be discharged at a discharge port 120d in line with the shaft 112 and the distal end bottom surface 115 can be used to spread the substance 200.

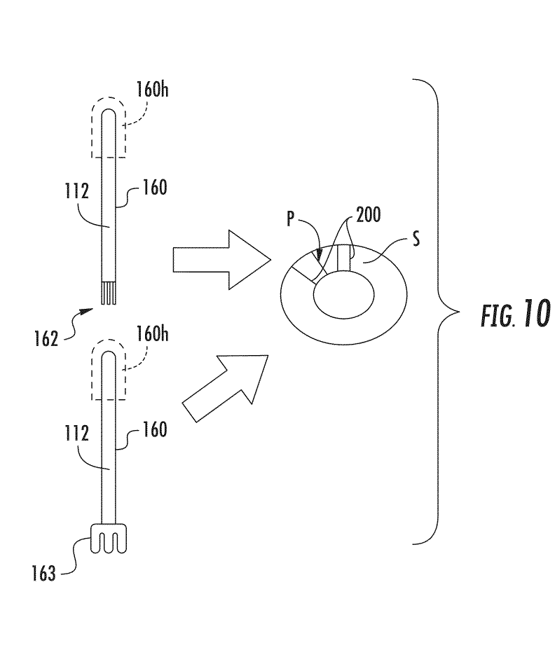

FIG. 7A illustrates that the tool 100 can be sterile and provided in a sterile package 150. FIG. 7A also illustrates that a package of one or more containers of tattoo substances 200 can be provided in support of the corneal tattoo procedures. Optionally, the containers 200 can be included in a package 155, which may be provided as a kit with the tool package 150 or as a separate package and may also be sterile. The package 155 can include a sterile board 210 for mixing different colors from different of the tattoo substance containers 200c to provide a custom color to match or more closely correspond to a patient's iris color(s).

FIG. 8 illustrates different color tattoo substances 200 on the board 210 that can be mixed by a clinician, typically the eye surgeon, prior to applying the corneal tattoo.

In some embodiments, as shown in FIG. 7B, a tattoo kit 200k can include a plurality of different color tattoo substances 200 (e.g., ink or pigments) in one color container package 200k with wells or sub-containers 200w (e.g., like a eye make-up kit of several colors of eye shadow, for example). The kit can include an electronic or paper instructions 200I with a range of colors 200c and associated color mixing instructions or recipes 200r for the different colors in the color kit 200k. The instructions 200I can be similar to a paint by number instruction with a desired color combination provided based on a name or row/column information of the colors in the kit 200k to generate a selected reference color using the color in a respective well or subcontainer of the multi-color tattoo substance kit or container (e.g., a plurality of dyes or pigments arranged by row/column for ease of selection).

The tool 100 can be provided as first and second tools, one with a clockwise configuration of the arcuate end 117e and the other with a counter clockwise configuration with the color kit 200k or color containers 200c or as a separate kit, tool or set of tools.

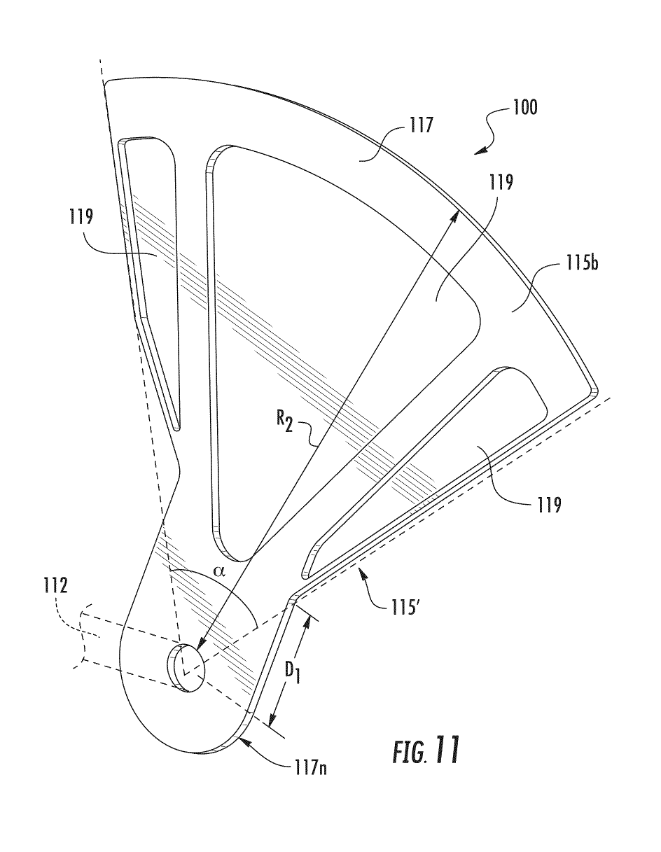

FIG. 9 illustrates that the laser system 10 can be in communication with or totally or partially have an onboard corneal tattoo module 75 that allows a user to select operational parameters for accessing a target corneal surface for the tattoo. Thus, the module 75 can be onboard the laser or laser system 10 or remotely accessible. The system 10 can also include or be in communication with a color match module 300. The system 10 can image the iris, using an imaging system that is part of the laser system 10 or a separate imaging system, and automatically match the imaged color to a color library 305. The system 10 can then provide the surgeon with color information for selecting appropriate dye colors, amounts of dye colors for mixing and the like, or a pre-made color dye of appropriate color, for a better color match via a UI or display.

This color information can be provided as a recipe to mix a set of different colors of tattoo substances to generate the patient color or the patient color can be provided pre-mixed in a container that can be purchased by product number. If remotely accessible, the corneal tattoo module 75 and/or the color match module 300 can be provided in one or more servers in a single site or a distributed “cloud” based network or system. A local computer can provide a portal for access to one or both modules 75, 300. In some embodiments, the color match module 300 can be provided as a web-based or online service and/or APP allowing a patient or other user to provide an image of the eye that is color matched for subsequent use in the treatment.

The terms “web-based,” “online” or “cloud-based” mean that the service is available using the World Wide Web (Internet), typically via at least one server to communicate with different users. The communication protocol can include hypertext transfer protocol (HTTP). The tattoo module 75 and/or color match module 300 can be provided using cloud computing which includes the provision of computational resources on demand via a computer network. The resources can be embodied as various infrastructure services (e.g., compute, storage, etc.) as well as applications, databases, file services, email, etc. In the traditional model of computing, both data and software are typically fully contained on the user's computer; in cloud computing, the user's computer may contain little software or data (perhaps an operating system and/or web browser), and may serve as little more than a display terminal for processes occurring on a network of external computers. A cloud computing service (or an aggregation of multiple cloud resources) may be generally referred to as the “Cloud.” Cloud storage may include a model of networked computer data storage where data is stored on multiple virtual servers, rather than being hosted on one or more dedicated servers.

As shown by way of example in FIG. 10, to form a more natural appearance after the first primary tattoo dye 200 is applied, one or more different color substances can be applied in a pattern P. The pattern P can be applied to the primary tattoo substance 200 using a different color as an overlayer on the primary cornea tattoo (e.g., tan, off white, black or the like) and this layer can be applied as an upper or over layer with a pattern forming tool 160 to form a radially extending striation pattern P, similar to the natural iris (FIGS. 3A, 3C, for example). The tool 160 can have a “paint” brush, smooth or course fiber(s) and/or a rake like end 162, 163 to form the striation pattern P. A nozzle or other device may also be able to dispense the second color substance and/or provide the striation pattern P. The striations can be a series of one or more of grooves, furrows or linear marks across the different color corneal tattoo.

The tool 100 and/or 160 can be single use, disposable. The tool 100, 160 can comprise a respective handle 100h, 160h that releasably engages the respective shaft 112. The tools 100, 160 can comprise molded integral or attached polymeric bodies (e.g., handles and shafts).

The tool 100 can be configured so that the shaft 112 and the distal end 115 can comprise a monolithic unitary molded body. In other embodiments the distal end may comprise a metal or ceramic and may be a separate member that attaches to the tool shaft.

The distal end 115 can be provided as a separate component that can engage the shaft 112 and can be provided in more than one size with more than one shape, width and/or radius of curvature to fit different patient eye requirements.

The shaft 112 and the distal end 115 of the tool 100 can be rigid or semi-rigid. The term “semi-rigid” means the distal end can flex somewhat but can substantially retain its shape. Where used, the distal end 162, 163 of striation tool 160 can be rigid, semi-rigid or flexible.

FIG. 12 illustrates exemplary actions to carry out a femtosecond laser assisted corneal tattoo procedure. The method can include selecting an incision location, size and depth for a corneal flap or pocket (block 400). Using the selected/input parameters, laser pulses can be transmitted to a lens of an eye of a patient to generate the corneal flap, channel or pocket. An internal corneal layer proximate an iris, e.g., in-line with a region of an iris over a pupil is accessed via the flap, channel or pocket. A corneal tattoo substance is applied to a target region of the corneal layer (block 420). The corneal tattoo substance can be manually pushed downward onto the cornea layer using a surgical tool to form a corneal tattoo (block 430).

In some embodiments, the corneal tattoo can be used to form a pinhole effect in a central cornea (e.g., for treating presbyopia) by placing the corneal tattoo substance as a “donut” like or annular shape with an open very small center space on the corneal tissue (block 440). The center space is thus surrounded by an opaque area of the applied corneal tattoo substance (pigment/dye). The outer diameter and/or radially extending width of the annular tattoo can be between 1-4 mm.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. In the claims, means-plus-function clauses, if used, are intended to cover the structures described herein as performing the recited function and not only structural equivalents but also equivalent structures. Therefore, it is to be understood that the foregoing is illustrative of the present invention and is not to be construed as limited to the specific embodiments disclosed, and that modifications to the disclosed embodiments, as well as other embodiments, are intended to be included within the scope of the appended claims. The invention is defined by the following claims, with equivalents of the claims to be included therein.

That which is claimed is:

1. A method of performing corneal tattoo surgery:
selecting an incision location, size and depth for a corneal flap or pocket; then
transmitting laser pulses to a lens of an eye of a patient to generate the corneal flap, channel or pocket; then
applying a corneal tattoo substance to a corneal layer and/or corneal tissue to form a corneal tattoo.

2. The method of claim 1, wherein the applying comprises manually pushing the corneal tattoo substance downward onto the cornea layer using a surgical tool to form a continuous layer or layers over a target area to form the corneal tattoo.

3. The method of claim 1, wherein the selecting is carried out by a user selecting a ring inner and outer diameter for a channel via a user interface in communication with a Femtosecond laser optical system, and wherein the transmitting laser pulses is carried out by the Femtosecond laser optical system.

4. The method of claim 1, wherein the corneal tattoo is applied in-line with a region of an iris over a pupil, and wherein the applying the corneal tattoo substance is carried out using at least one opaque, viscous dye having a clinically effective life of at least five years.

5. The method of claim 1, further comprising applying a striation pattern using a secondary dye onto the applied corneal tattoo to thereby generate a more natural looking iris for the corneal tattoo.

6. The method of claim 1, further comprising before the applying step, selecting a color corresponding to a primary color of the iris of the patient.

7. The method of claim 1, wherein the corneal tattoo is applied at a corneal depth of between 150 μm to 300 μm.

8. The method of claim 1, wherein the selecting and transmitting steps are carried out to generate an annular corneal channel, the annular corneal channel having an inner diameter of between 4-5 mm and an outer diameter that is between 1 mm to 3 mm larger than the inner diameter.

9. The method of claim 1, wherein the patient is treated for aniridia or iris trauma.

10. The method of claim 1, wherein the corneal tattoo is applied in an annular shape to form a pinhole effect for correcting presbyopia.

11. A surgical tool for corneal tattooing, comprising:
a handle with a shaft that is attached to a corneal applicator, the corneal applicator sized and configured to push against an exposed internal corneal layer of a cornea to distribute a viscous, opaque corneal tattoo substance.

12. The surgical tool of claim 11 in combination with at least one container of an opaque, viscous medical grade corneal tattoo substance.

13. The surgical tool of claim 11, wherein the corneal applicator comprises at least one depression or well for holding a quantity of the corneal tattoo substance.

14. The surgical tool of claim 11, wherein the corneal applicator has a thickness between 1 micron and 5 mm and extends off only a single side of the shaft.

15. The surgical tool of claim 13, further comprising a plurality of vertically extending through holes in the depression or well.

16. The surgical tool of claim 11, wherein the corneal applicator has a radially extending length and a circumferentially extending width associated with an outer perimeter edge, wherein the outer perimeter edge extends away from the shaft at an angle between 10-180 degrees.

17. The surgical tool of claim 11, wherein the corneal applicator is arcuate with opposing circumferentially spaced apart first and second ends, wherein the first end is attached to the shaft and the second end is a free end that resides circumferentially spaced apart from the shaft at between 90-180 degrees.

18. A surgical kit or set of components for treating an eye, comprising:
a surgical tool with a corneal applicator; and
at least one container of a medical grade corneal tattoo substance, wherein the corneal tattoo substance is formulated to be opaque and viscous.

19. The kit of claim 18, further comprising a striation tool configured to allow a user to apply a striation pattern over an opaque primary corneal tattoo that covers a continuous surface area over a target treatment site of a corneal layer to thereby form a more natural iris pattern for a corneal tattoo.

20. The kit of claim 18, further comprising a plurality of containers of the medical grade corneal tattoo substance, each of a different color.

21. The kit of claim 18, wherein the corneal applicator either (a) has a radially extending length and a circumferentially extending width associated with an outer perimeter edge, wherein the outer perimeter edge extends away from the shaft at an angle between 10-180 degrees or (b) is arcuate with opposing circumferentially spaced apart first and second ends, wherein the first end is attached to the shaft and the second end is a free end that resides circumferentially spaced apart from the shaft at between 90-180 degrees.

22. A femtosecond laser surgical system for eye treatments, comprising:
a femtosecond laser;
a controller in communication with the laser, the controller comprising or in communication with a corneal tattoo module, wherein the corneal tattoo module allows a user to select laser inputs including a corneal channel depth between 100 and 300 μm; and
a display having a user interface in communication with the controller, wherein the user interface allows a user to overlay a virtual ring over an image of an eye of a patient to identify a desired a ring size of an internal corneal layer in-line with a region of an iris over a pupil.

23. The system of claim 22, further comprising an imaging system in communication with the controller, wherein the system further comprising a color match module for evaluating an eye color of a patient based on an image taken using the imaging system and providing color information to a user for selecting an appropriate product or color for a corneal tattoo substance.

24. The system of claim 22, wherein the corneal tattoo module is configured to allow a user to apply an annular corneal tattoo to treat presbyopia.
